(19) United States
(12) Patent Application Publication
(10) Pub. No.: US 2022/0183783 A1
(43) Pub. Date: Jun. 16, 2022

(54) TRIGGER FINGER RELEASE PROCEDURE SINGLE USE, DISPOSABLE MEDICAL PROCEDURE KIT

(71) Applicant: WishBone Medical, Inc., Warsaw, IN (US)

(72) Inventor: Robert James von Seggern, Columbia City, IN (US)

(73) Assignee: WishBone Medical, Inc., Warsaw, IN (US)

(21) Appl. No.: 17/643,680

(22) Filed: Dec. 10, 2021

Related U.S. Application Data
(60) Provisional application No. 63/124,122, filed on Dec. 11, 2020.

(51) Int. Cl. A61B 50/30; A61B 50/33; A61L 2/20; A61L 2/08

(52) U.S. Cl. CPC A61B 50/3001; A61B 50/33; A61B 2050/005; A61L 2/08; A61L 2/206

(57) ABSTRACT

A kit for performing a medical procedure includes: a closed container; and a plurality of surgical items placed within the closed container. Each of the surgical items is sterilized and the container is closed to maintain sterility of the surgical items placed within. The surgical items include: a plurality of cutting instruments; at least one retractor; an analgesic; an antimicrobial solution; a surgical marker; at least one set of surgical gloves; a surgical drape; at least one length of suture; and a wound dressing.

### CROSS REFERENCE TO RELATED APPLICATIONS

This is a non-provisional application based upon U.S. provisional patent application Ser. No. 63/124,122, entitled “Trigger Finger Release single use, disposable medical procedure kit”, filed Dec. 11, 2020, which is incorporated herein by reference.

### BACKGROUND OF THE INVENTION

### 1. Field of the Invention

The present invention relates to medical instruments, and, more particularly, to medical procedure kits for orthopaedic surgeries.

### 2. Description of the Related Art

When a tendon becomes adhered so the associated muscles and/or bones cannot move properly, a release procedure may be needed. One common release procedure is referred to as a “trigger finger release procedure” to release a finger. There are numerous surgical techniques to perform a trigger finger release procedure, depending upon the preference of healthcare provider. All these techniques require specialized surgical instruments, implants, and operating room procedure setup. The instruments are typically collected by a nurse the day of the procedure and sterilized for the operation. The potential risks associated with this practice are defective sterilization, an incomplete instrument/implant kit, damaged instrumentation, increased time in the operating room, and surgical delay. These risks can lead to a non-optimized surgery.

What is needed in the art is a way to address some of the issues with known release procedures.

### SUMMARY OF THE INVENTION

The present invention provides a kit for a medical procedure that includes all the items needed to perform the procedure in a closed container that keeps the items sterilized.

The invention in one form is directed to a kit for performing a medical procedure, the kit including a closed container and a plurality of surgical items placed within the closed container. Each of the surgical items is sterilized and the container is closed to maintain sterility of the surgical items placed within. The surgical items include: a plurality of cutting instruments; at least one retractor; an analgesic; an antimicrobial solution; a surgical marker; at least one set of surgical gloves; a surgical drape; at least one length of suture; and a wound dressing.

The invention in another form is directed to a method of preparing a ready-to-use kit for a medical procedure, the method includes: sterilizing a plurality of surgical items; placing the surgical items in a container; and closing the container so the surgical items maintain sterility within the container. The surgical items include: a plurality of cutting instruments; at least one retractor; an analgesic; an antimicrobial solution; a surgical marker; at least one set of surgical gloves; a surgical drape; at least one length of suture; and a wound dressing.

The invention in yet another form is directed to a method of performing a tenolysis procedure, the method includes: opening a container of a kit, the container holding a plurality of surgical items that are sterilized; removing at least some of the surgical items from the container; forming an incision in a patient using only removed surgical items; and releasing a tendon of the patient using only removed surgical items. The surgical items include: a plurality of cutting instruments; at least one retractor; an analgesic; an antimicrobial solution; a surgical marker; at least one set of surgical gloves; a surgical drape; at least one length of suture; and a wound dressing.

An advantage of the present invention is the kit is equipped with all the sterilized surgical items that a surgeon will need to perform a release procedure, eliminating the need for a medical professional to assemble the items together and sterilize the items.

Another advantage is the kit can be used as an “off-the-shelf” item that is stored at a medical facility, ensuring that all of the items needed to perform the procedure are available on the day of surgery.

### BRIEF DESCRIPTION OF THE DRAWINGS

The above-mentioned and other features and advantages of this invention, and the manner of attaining them, will become more apparent and the invention will be better understood by reference to the following description of embodiments of the invention taken in conjunction with the accompanying drawings, wherein:

FIG. 1 is a perspective view of an exemplary embodiment of the contents of a kit formed according to the present invention;

FIG. 2 is a sectional view of an exemplary embodiment of a kit formed according to the present invention;

FIG. 3 is a flow chart illustrating an exemplary embodiment of a method of forming a kit for a medical procedure according to the present invention; and

FIG. 4 is a flow chart illustrating an exemplary embodiment of a method of performing a tenolysis procedure according to the present invention.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION OF THE INVENTION

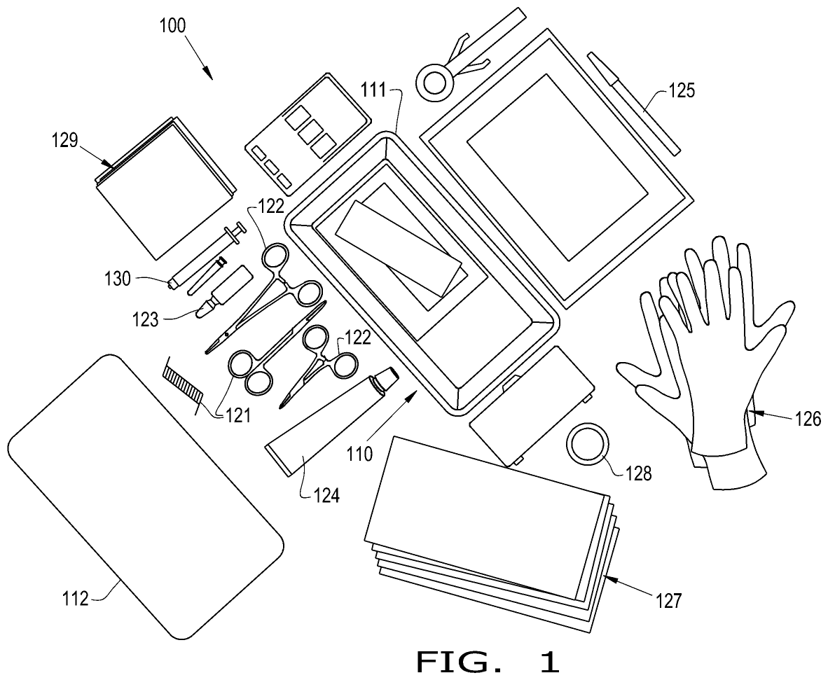

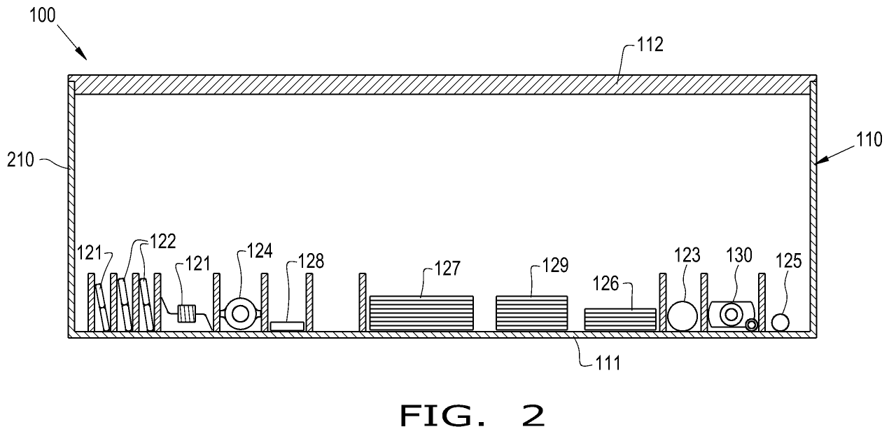

Referring now to the drawings, and more particularly to FIGS. 1-2, there is shown an exemplary embodiment of a kit 100 for performing a medical procedure which generally includes a container 110 and a plurality of surgical items 121, 122, 123, 124, 125, 126, 127, 128, 129 placed in the container 110. Each of the surgical items 121, 122, 123, 124, 125, 126, 127, 128, 129 is sterilized and the container 110 is closed to maintain sterility of the surgical items 121, 122, 123, 124, 125, 126, 127, 128, 129 placed within. As used herein, “sterile” and “sterility” should be understood according to the commonly understood definitions of the terms referring to a near-total, if not total, absence of viable microorganisms on the surgical items 121, 122, 123, 124, 125, 126, 127, 128, 129. Many sterilization techniques are known, and the surgical items 121, 122, 123, 124, 125, 126, 127, 128, 129 may be sterilized according to any suitable sterilization technique. In some embodiments, each of the surgical items 121, 122, 123, 124, 125, 126, 127, 128, 129 is a “single-use” item that is intended to be disposed of after the medical procedure is performed and/or is not designed to be re-sterilized. In some embodiments, at least some of the surgical items 121, 122, 123, 124, 125, 126, 127, 128, 129 are individually held sterile in packaging when placed in the container 110.

Since the surgical items 121, 122, 123, 124, 125, 126, 127, 128, 129 maintain sterility within the container 110, the surgical items 121, 122, 123, 124, 125, 126, 127, 128, 129 can be brought into a sterile field, such as an operating room, and used without needing to be sterilized again. In some embodiments, the container 110 is also sterilized so the container 110 does not also need to be sterilized before being brought into a sterile field. The container 110 may, for example, be closed with the surgical items 121, 122, 123, 124, 125, 126, 127, 128, 129 placed within and then placed in a sterile packaging 210, such as a sterile bag, to maintain sterility of the container 110 as well. The container 110 may be stored in the sterile packaging 210 at a healthcare facility, such as a hospital, and then removed from the sterile packaging 210 shortly before being brought into the sterile field, virtually eliminating the need to sterilize the container 110 and/or any of the surgical items 121, 122, 123, 124, 125, 126, 127, 128, 129 at the healthcare facility.

The surgical items 121, 122, 123, 124, 125, 126, 127, 128, 129 placed within the container 110 are all the surgical items 121, 122, 123, 124, 125, 126, 127, 128, 129 that a surgeon and/or other healthcare provider may need to use during a medical procedure, such as a tenolysis procedure. The surgical items 121, 122, 123, 124, 125, 126, 127, 128, 129 include: a plurality of cutting instruments 121, illustrated as scissors and a tendon knife; at least one retractor 122, illustrated as a Ragnell retractor; an analgesic 123, such as lidocaine; an antimicrobial solution 124, such as bacitracin; a surgical marker 125, such as a skin-safe pen; at least one set of surgical gloves 126, which may be one set of latex gloves and one set of nitrile gloves; a surgical drape 127; at least one length of suture 128, such as a length of 3-0 suture, a length of 5-0 suture, and/or a length of 6-0 suture; and a wound dressing 129, which may include gauze and/or a light pressure dressing. In some embodiments, at least one hypodermic needle 130 may also be provided to, for example, administer the analgesic 123. An operating team may thus take the kit 100 into a sterile field knowing that all of the surgical items needed for a tenolysis procedure are in the kit 100 and sterilized for the procedure. It should be appreciated that the surgical items 121, 122, 123, 124, 125, 126, 127, 128, 129, 130 provided in the container 100 may be adjusted, as needed, to provide all of the instruments needed for a particular procedure.

In some embodiments, as previously described, the container 110 is sterilized as well so the container 110 can be brought into the sterile field. In some embodiments, the container 110 includes a tray 111 that is closed by a lid 112 coupled to the tray 111. The tray 111 may be formed to hold all of the surgical items 121, 122, 123, 124, 125, 126, 127, 128, 129, 130 in a sorted manner so the surgical team knows where to expect each individual surgical item 121, 122, 123, 124, 125, 126, 127, 128, 129, 130. In some embodiments, the tray 111 may include slots or other features to firmly hold each surgical item 121, 122, 123, 124, 125, 126, 127, 128, 129, 130. The tray 111 may also include markings or other indicators that convey where each of the surgical items 121, 122, 123, 124, 125, 126, 127, 128, 129, 130 is meant to be held on the tray 111. The lid 112 is configured to form a liquid-proof, and in some embodiments hermetic, seal with the tray 111 when closed to substantially prevent the ingress of contaminants into the closed container 110. In some embodiments, the closed container 110 may have a positive pressure established within the container 110 when the surgical items 121, 122, 123, 124, 125, 126, 127, 128, 129, 130 are placed in the container 110, further reducing the risk of contamination within the container 110.

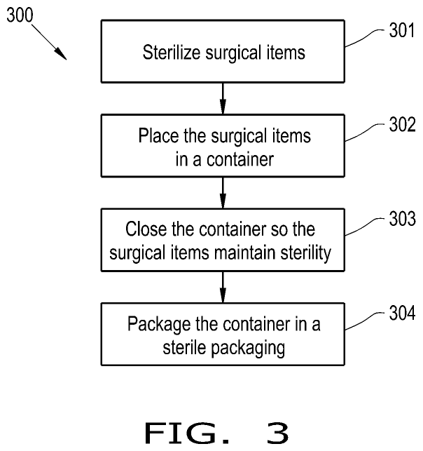

Referring now to FIG. 3, an exemplary embodiment of a method 300 of preparing a ready-to-use kit for a medical procedure, such as the kit 100, is illustrated. The method 300 includes sterilizing 301 a plurality of surgical items 121, 122, 123, 124, 125, 126, 127, 128, 129, 130; placing 302 the surgical items 121, 122, 123, 124, 125, 126, 127, 128, 129, 130 in a container 110; and closing 303 the container 110 so the surgical items 121, 122, 123, 124, 125, 126, 127, 128, 129, 130 maintain sterility within the container 110. Closing 303 the container 110 may include, for example, closing the tray 111 with the lid 112, as previously described.

In some embodiments, the container 110 is also sterilized, which may be performed concurrently with sterilizing 301 the surgical items 121, 122, 123, 124, 125, 126, 127, 128, 129, 130 or separately. For example, the surgical items 121, 122, 123, 124, 125, 126, 127, 128, 129, 130 may be placed in the container 110 and then the surgical items 121, 122, 123, 124, 125, 126, 127, 128, 129, 130 and the container 110 may be placed in a sterilization chamber for sterilization. Sterilization may be performed in many different ways. For example, sterilization may include irradiation, autoclaving, and/or ethylene oxide (EtO) sterilization of one or more of the surgical items 121, 122, 123, 124, 125, 126, 127, 128, 129, 130 and/or the container 110. In some embodiments, multiple sterilization techniques may be performed, e.g., some of the surgical items 121, 122, 123, 124, 125, 126, 127, 128, 129, 130 may be sterilized using autoclaving while others are sterilized using EtO sterilization. Even further, some of the surgical items 121, 122, 123, 124, 125, 126, 127, 128, 129, 130 and/or the container 110 may be sterilized using two different sterilization techniques, e.g., by irradiation followed by Et0 sterilization. It should thus be appreciated that the surgical items 121, 122, 123, 124, 125, 126, 127, 128, 129, 130 and, in some embodiments, the container 110 can be sterilized in a variety of ways. The method 300 may further include packaging 304 the closed container 110 holding the sterilized surgical items 121, 122, 123, 124, 125, 126, 127, 128, 129, 130 in a sterile packaging, such as a sterile bag, to maintain sterility of the container 110 as well. Therefore, it should be appreciated that the kit 100 may be provided by sterilizing and packaging the surgical items 121, 122, 123, 124, 125, 126, 127, 128, 129, 130 and the container 110 so all elements of the kit 100 are sterile and ready-to-use in a sterile field.

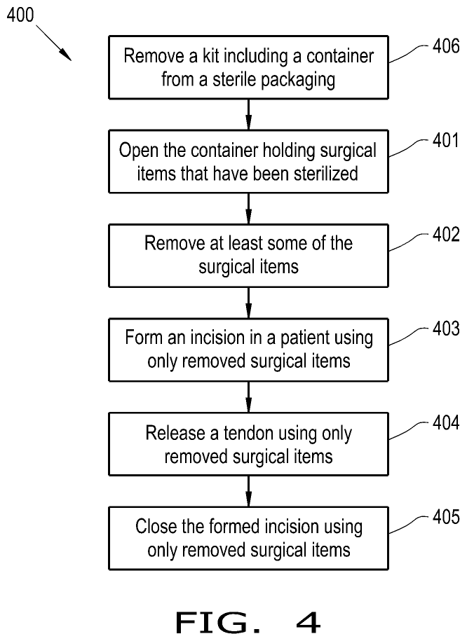

In some embodiments, and referring now to FIG. 4, a method 400 of performing a tenolysis procedure is provided according to the present invention. The method 400 includes: opening 401 the container 110 of the kit 100, the container 110 holding the surgical items 121, 122, 123, 124, 125, 126, 127, 128, 129, 130 that have been sterilized; removing 402 at least some of the surgical items 121, 122, 123, 124, 125, 126, 127, 128, 129, 130 from the container 110; forming 403 an incision in a patient using only removed surgical items 121, 122, 123, 124, 125, 126, 127, 128, 129, 130; and releasing 404 a tendon of the patient using only removed surgical items 121, 122, 123, 124, 125, 126, 127, 128, 129, 130. As used here, the term “using only removed surgical items” refers to a surgeon and/or other healthcare provider(s) not using any tools other than those provided in the kit 100. Opening 401 the container 110 may include opening a lid 112 that is coupled to a tray 111 of the container 110. In some embodiments, the container 110 is sterilized prior to opening 401 the container 110, as previously described. In some embodiments, all of the surgical items 121, 122, 123, 124, 125, 126, 127, 128, 129, 130 are removed from the container 110, but may not necessarily be used to release 404 the tendon. Releasing 404 the tendon may include, for example, cutting an A1 pulley of the patient to perform what is known in the art as a “trigger finger release.” It should be appreciated, however, that releasing 404 the tendon can be done by cutting and/or other manipulating other anatomical structures of the patient. The method 400 may further including closing 405 the formed incision using only removed surgical items 121, 122, 123, 124, 125, 126, 127, 128, 129, 130. When the kit 100 is provided in a sterile packaging 210, as previously described, the method 400 further includes removing 406 the kit 100 from the sterile packaging 210 prior to opening 401 the container 110. Thus, the method 400 may be provided to perform a tenolysis procedure using the kit 100, which contains all of the pre-sterilized surgical items 121, 122, 123, 124, 125, 126, 127, 128, 129, 130 needed to perform the procedure.

From the foregoing, it should be appreciated that the kit 100 and methods 300, 400 provided according to the present invention offer multiple benefits. Sterile packed, single-use, disposable procedure kits can be sold to hospitals and surgery centers and can easily reduce inventory by a factor of 10 compared to the old model.

With an emphasis today on cost reduction and improved patient outcomes, as well as the ever-present goal to improve operating room (OR) efficiencies and reduce reprocessing needs, more surgeons are moving toward single-use, disposable instruments. Sterile packed, single use kits, such as the kit 100, promote ease of use and improve operating room turnover. The Operating Room (OR) is the largest generator of revenue for hospitals and accounts for approximately 40% of total revenue. The OR also comprises up to 56% of overall hospital supply expense. It has been demonstrated that surgical delays as brief as 10 minutes may cost a hospital more than $130,000 in staffing costs alone per year. Efficiency in the OR is paramount.

The simplified procedure kits provided according to the present invention are designed to drive repeatable procedures with all instruments and implants required for a particular procedure. They may be arranged in a way that allows the surgeon to focus on the patient without distraction from the back table or the need to search for a particular item needed. Additionally, the “old model” of reusable trays requires a 3.5-hour sanitation cycle at a cost of $285 per procedure. The sterilization cycle prohibits back-to-back surgeries which surgeons and hospitals prefer.

Sterile packed, single-use kits provided according to the present invention allow for back-to-back surgeries by eliminating the sanitation cycle. Finally, reprocessing/sterilization of instruments is also a time-consuming and costly hassle for both hospitals and ambulatory surgical centers.

Single-use instruments and other items, such as the surgical items 121, 122, 123, 124, 125, 126, 127, 128, 129, 130 provided according to the present invention, significantly reduce the possibility of spreading bacterial infections and so-called “superbugs” from equipment that has not been completely sanitized. The U.S. Food and Drug Administration (FDA) has announced one of its regulatory science priorities is the reduction of healthcare-associated infections. One infection is estimated to cost a hospital $45,000 per occurrence. The FDA is especially concerned about cleaning, re-sterilization, handling, and re-calibration of instruments. This is in response to the serious health risks posed by superbugs and other microorganisms associated with reprocessing/cleaning and re-sterilization of duodenoscopes and other devices, including reusable orthopedic instruments and devices. As a result, the FDA favors sterile-packed, single-use (disposable) instrument alternatives, as they eliminate this risk and are also in pristine condition and calibration for use in a medical procedure. As can be appreciated from the foregoing, the kit 100 provided according to the present invention can allow healthcare providers to perform medical procedures while avoiding many of the disadvantages associated with the known ways of providing surgical instruments and other items for medical procedures in the OR.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A kit for performing a medical procedure, the kit comprising:
a closed container; and
a plurality of surgical items placed within the closed container, each of the surgical items being sterilized and the container being closed to maintain sterility of the surgical items placed within, the surgical items comprising:
a plurality of cutting instruments;
at least one retractor;
an analgesic;
an antimicrobial solution;
a surgical marker;
at least one set of surgical gloves;
a surgical drape;
at least one length of suture; and
a wound dressing.

2. The kit of claim 1, wherein the at least one retractor comprises a Ragnell retractor.

3. The kit of claim 1, wherein the plurality of cutting instruments comprises a knife and a pair of scissors.

4. The kit of claim 1, further comprising at least one hypodermic needle.

5. The kit of claim 1, wherein the closed container is sterilized.

6. The kit of claim 1, wherein the closed container comprises a tray closed by a lid.

7. A method of preparing a kit for a medical procedure, the method comprising:
sterilizing a plurality of surgical items, the plurality of surgical items comprising:
a plurality of cutting instruments;
at least one retractor;
an analgesic;
an antimicrobial solution;
a surgical marker;
at least one set of surgical gloves;
a surgical drape;
at least one length of suture; and
a wound dressing;
placing the surgical items in a container; and
closing the container so the surgical items maintain sterility within the container.

8. The method of claim 7, further comprising sterilizing the container.

9. The method of claim 8, wherein sterilizing the surgical items and sterilizing the container are performed concurrently.

10. The method of claim 7, wherein the sterilizing comprises at least one of irradiation, autoclaving, or ethylene oxide sterilization.

11. The method of claim 7, wherein sterilizing the surgical items occurs before placing the surgical items in the container.

12. The method of claim 7, wherein the container comprises a tray and a lid coupled to the tray and closing the container comprises closing the tray with the lid.

13. The method of claim 7, further comprising packaging the closed container in a sterile packaging.

14. A method of performing a tenolysis procedure, the method comprising:
opening a container of a kit, the container holding a plurality of surgical items that are sterilized, the surgical items comprising:
a plurality of cutting instruments;
at least one retractor;
an analgesic;
an antimicrobial solution;
a surgical marker;
at least one set of surgical gloves;
a surgical drape;
at least one length of suture; and
a wound dressing;
removing at least some of the surgical items from the container;
forming an incision in a patient using only removed surgical items; and
releasing a tendon of the patient using only removed surgical items.

15. The method of claim 14, further comprising closing the formed incision using only removed surgical items.

16. The method of claim 14, further comprising removing the kit from a sterilized packaging prior to opening the container.

17. The method of claim 14, wherein opening the container comprises opening a lid that is coupled to a tray of the container.

18. The method of claim 14, wherein the container is sterilized prior to opening the container.

19. The method of claim 14, wherein all of the surgical items are removed from the container.

20. The method of claim 14, wherein releasing the tendon comprises cutting an Al pulley of the patient.
